(19) United States
(12) Patent Application Publication
(10) Pub. No.: US 2019/0082983 A1
(43) Pub. Date: Mar. 21, 2019

(54) FINGER CUFF ASSEMBLY HAVING A SINGLE-SIZED INFLATABLE BLADDER

(71) Applicant: Edwards Lifesciences Corporation, Irvine, CA (US)

(72) Inventors: Olaf Schraa, Amsterdam (NL); Peiyuan Li, Amsterdam (NL)

(21) Appl. No.: 16/130,887

(22) Filed: Sep. 13, 2018

Related U.S. Application Data
(60) Provisional application No. 62/560,440, filed on Sep. 19, 2017.

(51) Int. Cl. A61B 5/0225; A61B 5/022; A61B 5/021

(52) U.S. Cl. CPC A61B 5/02255; A61B 5/02241; A61B 2562/0247; A61B 5/02225; A61B 5/02141

(57) ABSTRACT

Disclosed is a finger cuff assembly that is attachable to a patient's finger to be used in measuring the patient's blood pressure by a blood pressure measurement system. The finger cuff assembly includes a plethysmograph. The plethysmograph includes a light emitting diode (LED)—photodiode (PD) pair that aids in measuring the patient's blood pressure by the blood pressure measurement system. The finger cuff assembly further includes an outer ring and a bladder. The bladder is contained within the outer ring and includes an inflatable inner portion and a finger cavity. The patient's finger with the plethysmograph surrounding the patient's finger may be received and surrounded within the finger cavity of the bladder.

### CROSS-REFERENCE TO RELATED APPLICATIONS

This application claims priority to U.S. Provisional Application No. 62/560,440, filed Sep. 19, 2017, the contents of which are incorporated herein by reference.

### BACKGROUND

### Field

Embodiments of the invention relate generally to non-invasive blood pressure measurement. More particularly, embodiments of the invention relate to a finger cuff assembly for blood pressure measurement.

### Relevant Background

Volume clamping is a technique for non-invasively measuring blood pressure in which an external pressure is applied to a patient's finger in such a manner that arterial pressure may be balanced by a time varying pressure to maintain a constant arterial volume. In a properly fitted and calibrated system, the applied time varying pressure is equal to the arterial blood pressure in the finger. The applied time varying pressure may be measured to provide a reading of the patient's arterial blood pressure.

This may be accomplished by a finger cuff that is arranged around a finger of a patient. The finger cuff may include an infrared light source, an infrared sensor, and an inflatable bladder. The infrared light may be sent through the finger in which a finger artery is present. The infrared sensor picks up the infrared light and the amount of infrared light registered by the sensor may be inversely proportional to the artery diameter and indicative of the pressure in the artery.

In the finger cuff implementation, by inflating the bladder in the finger cuff, a pressure is exerted on the finger artery. If the pressure is high enough, it will compress the artery and the amount of light registered by the sensor will increase. The amount of pressure necessary in the inflatable bladder to compress the artery is dependent on the blood pressure. By controlling the pressure of the inflatable bladder such that the diameter of the finger artery is kept constant, the blood pressure may be monitored in very precise detail as the pressure in the inflatable bladder is directly linked to the blood pressure. In a typical present day finger cuff implementation, a volume clamp system is used with the finger cuff. The volume clamp system typically includes a pressure generating system and a regulating system that includes: a pump, a valve, and a pressure sensor in a closed loop feedback system that are used in the measurement of the arterial volume. To accurately measure blood pressure, the feedback loop provides sufficient pressure generating and releasing capabilities to match the pressure oscillations of the patient's blood pressure.

Due to the differences in patients' physical conditions (i.e., differently sized fingers), differently-sized finger cuffs (e.g., large, medium, small, etc.) having differently-sized bladders are currently required in order to accommodate large, medium and small fingers, to obtain accurate measurements. However, producing such bladders in different sizes may increase the complexity of product manufacturing and logistics management. Further, from a healthcare provider's standpoint, the healthcare provider needs to be cautious in selecting an appropriate finger cuff size for the patient in order to obtain effective measurements. Accordingly, it would be beneficial to have a finger cuff with a single-sized or one-size-fits-all inflatable bladder.

### SUMMARY

Embodiments of the invention may relate to a finger cuff assembly that is attachable to a patient's finger to be used in measuring the patient's blood pressure by a blood pressure measurement system. The finger cuff assembly includes a plethysmograph. The plethysmograph includes a light emitting diode (LED)—photodiode (PD) pair that aids in measuring the patient's blood pressure by the blood pressure measurement system. The finger cuff assembly further includes an outer ring and a bladder. The bladder is contained within the outer ring and includes an inflatable inner portion and a finger cavity. The patient's finger with the plethysmograph surrounding the patient's finger may be received and surrounded within the finger cavity of the bladder.

### BRIEF DESCRIPTION OF THE DRAWINGS

FIG. 1 is a diagram of an example of a blood pressure measurement system according to one embodiment.

FIG. 2 is a diagram illustrating an example of a finger cuff assembly according to one embodiment.

FIGS. 3A-3C are diagrams illustrating cross-sectional views of the finger cuff assembly according to one embodiment.

FIG. 4 is a block diagram illustrating an example environment in which embodiments of the invention may be practiced.

### DETAILED DESCRIPTION

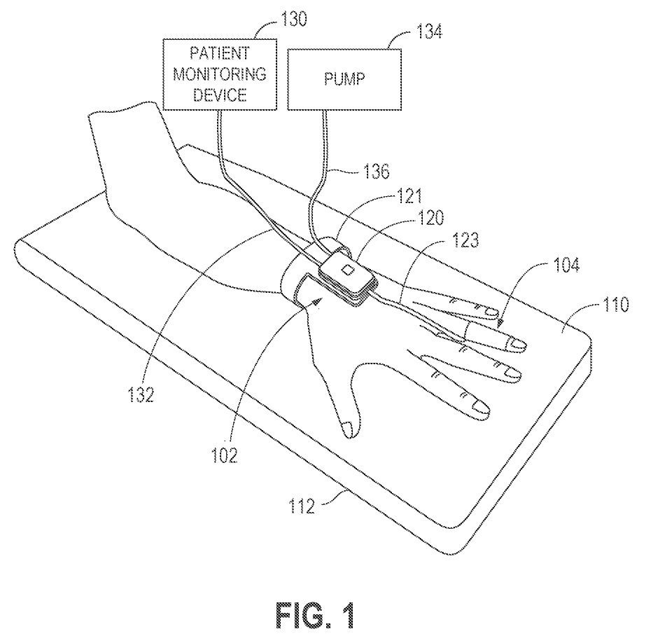

With reference to FIG. 1, which illustrates an example of a blood pressure measurement system according to one embodiment, a blood pressure measurement system 102 that includes a finger cuff 104 that may be attached to a patient's finger and a blood pressure measurement controller 120, which may be attached to the patient's body (e.g., a patient's wrist or hand) is shown.

The blood pressure measurement system 102 may further be connected to a patient monitoring device 130, and, in some embodiments, a pump 134. Further, finger cuff 104 may include a bladder (not shown) and an LED-PD pair (not shown), which are conventional for finger cuffs.

In one embodiment, the blood pressure measurement system 102 may include a pressure measurement controller 120 that includes: a small internal pump, a small internal valve, a pressure sensor, and control circuitry. In this embodiment, the control circuitry may be configured to: control the pneumatic pressure applied by the internal pump to the bladder of the finger cuff 104 to replicate the patient's blood pressure based upon measuring the plethysmograph signal received from the LED-PD pair of the finger cuff 104. Further, the control circuitry may be configured to: control the opening of the internal valve to release pneumatic pressure from the bladder; or the internal valve may simply be an orifice that is not controlled. Additionally, the control circuitry may be configured to: measure the patient's blood pressure by monitoring the pressure of the bladder based upon the input from a pressure sensor, which should be the same as patient's blood pressure, and may display the patient's blood pressure on the patient monitoring device 130.

In another embodiment, a conventional pressure generating and regulating system may be utilized, in which, a pump 134 is located remotely from the body of the patient. In this embodiment, the blood pressure measurement controller 120 receives pneumatic pressure from remote pump 134 through tube 136 and passes on the pneumatic pressure through tube 123 to the bladder of finger cuff 104. Blood pressure measurement device controller 120 may also control the pneumatic pressure (e.g., utilizing a controllable valve) applied to the finger cuff 104 as well as other functions. In this example, the pneumatic pressure applied by the pump 134 to the bladder of finger cuff 104 to replicate the patient's blood pressure based upon measuring the plethysmograph signal received from the LED-PD pair of the finger cuff 104 and measuring the patient's blood pressure by monitoring the pressure of the bladder may be controlled by the blood pressure measurement controller 120 and/or a remote computing device and/or the pump 134 and/or the patient monitoring device 130 to implement the volume clamping method. In some embodiments, a blood pressure measurement controller 120 is not used at all and there is simply a connection from tube 136 from a remote pump 134 including a remote pressure regulatory system to finger cuff 104, and all processing for the pressure generating and regulatory system, data processing, and display is performed by a remote computing device.

Continuing with this example, as shown in FIG. 1, a patient's hand may be placed on the face 110 of an arm rest 112 for measuring a patient's blood pressure with the blood pressure measurement system 102. The blood pressure measurement controller 120 of the blood pressure measurement system 102 may be coupled to a bladder of the finger cuff 104 in order to provide pneumatic pressure to the bladder for use in blood pressure measurement. Blood pressure measurement controller 120 may be coupled to the patient monitoring device 130 through a power/data cable 132. Also, in one embodiment, as previously described, in a remote implementation, blood pressure measurement controller 120 may be coupled to a remote pump 134 through tube 136 to receive pneumatic pressure for the bladder of the finger cuff 104. The patient monitoring device 130 may be any type of medical electronic device that may read, collect, process, display, etc., physiological readings/data of a patient including blood pressure, as well as any other suitable physiological patient readings. Accordingly, power/data cable 132 may transmit data to and from patient monitoring device 130 and also may provide power from the patient monitoring device 130 to the blood pressure measurement controller 120 and finger cuff 104.

As can be seen in FIG. 1, in one example, the finger cuff 104 may be attached to a patient's finger and the blood pressure measurement controller 120 may be attached on the patient's hand or wrist with an attachment bracelet 121 that wraps around the patient's wrist or hand. The attachment bracelet 121 may be metal, plastic, Velcro, etc. It should be appreciated that this is just one example of attaching a blood pressure measurement controller 120 and that any suitable way of attaching a blood pressure measurement controller to a patient's body or in close proximity to a patient's body may be utilized and that, in some embodiments, a blood pressure measurement controller 120 may not be used at all. It should further be appreciated that the finger cuff 104 may be connected to a blood pressure measurement controller described herein, or a pressure generating and regulating system of any other kind, such as a conventional pressure generating and regulating system that is located remotely from the body of the patient (e.g., a pump 134 located remotely from a patient). Any kind of pressure generating and regulating system can be used, including but not limited to the blood pressure measurement controller, and may be described simply as a pressure generating and regulating system that may be used with a finger cuff 104 including an LED-PD pair and a bladder to implement the volume clamping method.

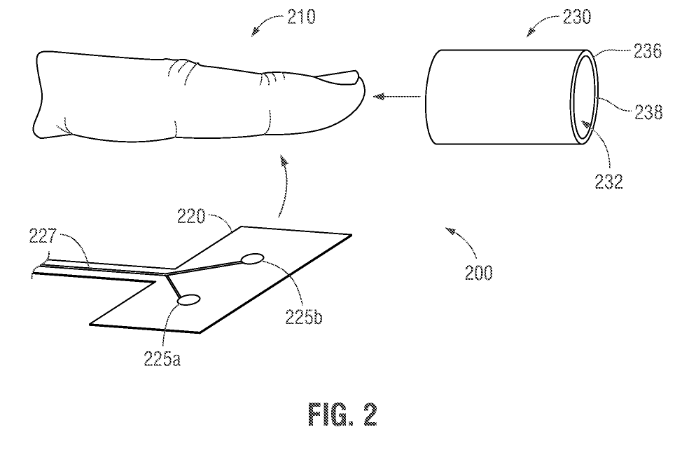

FIG. 2 is a diagram illustrating an example of a finger cuff assembly according to one embodiment. In some embodiments, finger cuff assembly 200 may be finger cuff 104, as previously described in FIG. 1. Referring to FIG. 2, finger cuff assembly 200 may be placed around a patient's finger 210. Finger cuff assembly 200 may include a plethysmograph 220 having an LED-PD pair 225a-b and an inflatable bladder 230. In some embodiments, the plethysmograph 220 may be separately applied or placed (e.g., by a healthcare provider) on or around the patient's finger 210 (e.g., middle phalanx of an index, middle, or ring finger) such that the plethysmograph 220 abuts against finger 210 (i.e., the patient's skin) in order to obtain the plethysmogram from the finger 210. To facilitate the placement of the plethysmograph 220 on the finger 210, an end, side, or other portion of the plethysmograph 220, on the interior, may include removable or reusable adhesive material (i.e., an adhesive layer) so that the plethysmograph 220 can be removably attached to the finger 210. It should be appreciated that this is just an example of an attachment mechanism and that any suitable type may be utilized. In one embodiment, plethysmograph 220 may be of extended length and approximately rectangular-shaped. In one embodiment, the plethysmograph 220 may be thin and of opaque, elastic material (e.g., opaque foil). The plethysmograph 220, for example, may be of a color white, black, or metallic (e.g., aluminum) so that it is opaque for infrared (IR) light. In one embodiment, the plethysmograph 220 may be a disposable plethysmograph, for example as discussed in U.S. Provisional Patent Application Ser. No. 62/555,425, filed on Sep. 7, 2017 and entitled MODULAR FINGER CUFF, the disclosure of which is incorporated herein by reference for all purposes.

As shown in FIG. 2, inflatable bladder 230 may include an outer ring 236 and an inflatable inner portion 238. The inflatable inner portion 238 of the bladder is contained within the outer ring 236. The outer ring 236 may surround the inflatable inner portion 238 of the bladder, with the inflatable inner portion 238 permanently or removably attaching to the interior of the outer ring 236. The outer ring 236 may be approximately cylindrically-shaped and the inflatable inner portion 238 may be approximately conically-shaped. The inflatable bladder 230 may further include a finger cavity 232 that is approximately oval-shaped for insertion of the patient's finger 210 into the bladder 230, for example by the healthcare provider. In some embodiments, after placing the plethysmograph 220 on the finger 210, the finger 210 (along with the plethysmograph 220) may be inserted through finger cavity 232 of bladder 230 such that the inflatable inner portion 238 of the bladder, effectively surrounds and/or abuts against the plethysmograph 220 and/or finger 210. In some embodiments, the inflatable inner portion 238 of bladder 230 may be of flexible and/or non-elastic material. In one embodiment, outer ring 236 of bladder 230 may be of rigid or stiff material. In some embodiments, the bladder 230 may be reusable. In some embodiments, the LED of LED-PD pair 225a-b pair may be an organic light emitting diode (OLED).

Continuing with reference to FIG. 2, the LED-PD pair 225a-b may be coupled or connected to a cable 227 through a connector (not shown), which may be attached to plethysmograph 220, to provide power to and receive data (i.e., electrical signals) from the LED-PD pair 225a-b. Additionally, the bladder 230 may be coupled or connected to a tube (not shown) to provide pneumatic pressure to the inflatable inner portion 238.

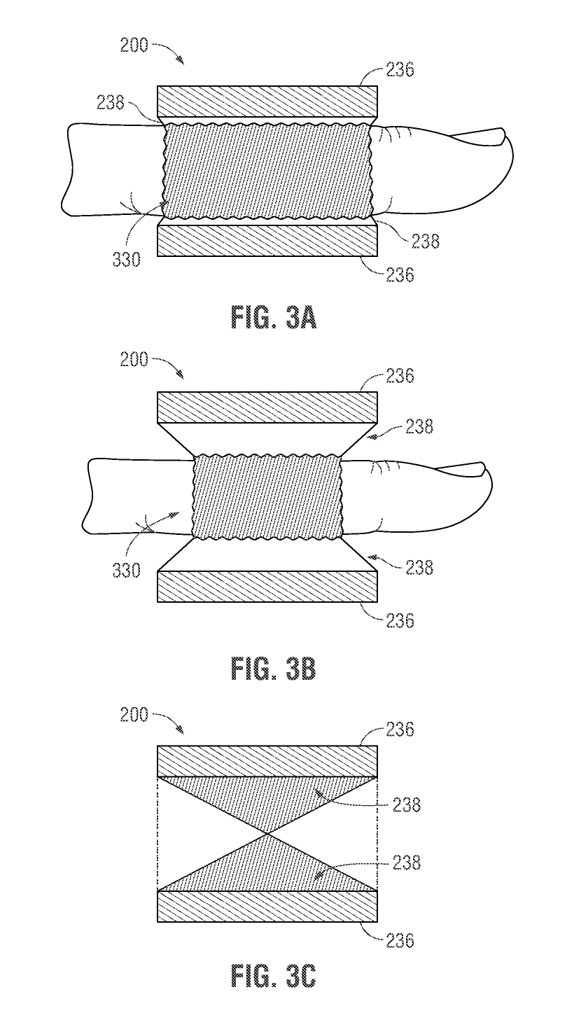

FIGS. 3A-3C are diagrams illustrating cross-sectional views of finger cuff assembly 200 according to one embodiment. With reference to FIGS. 3A-3C, the finger cuff assembly 200 may be placed around two fingers with different sizes (e.g., different finger diameters or circumferences), with a larger finger being illustrated in FIG. 3A and a smaller finger being illustrated in FIG. 3B. FIG. 3C shows the inflatable inner portion 238 of the bladder being inflated without a finger therein. As an example, it may be approximately conically-shaped. As can be seen, pressure (e.g., pneumatic pressure) may be applied to each of the two fingers from the inflatable inner portion 238 of the bladder at a pressurized (or contact) area 330 of the finger. In one embodiment, the pressure may be applied from inflatable inner portion 238, through a plethysmograph (not shown) and to the finger (as the plethysmograph may be already attached to the finger—as previously described). The pressurized area 330 between the finger and the inflatable inner portion 238 may vary, depending upon the size of the finger. For example, in FIG. 3A, the length of the pressurized area 330 of the larger finger is greater than the length of the pressurized area 330 of the smaller finger in FIG. 3B. The length of the pressured area 330, therefore, may be variable based upon the size of the patient's finger. In one embodiment, the shape (or form) of the inflatable inner portion 238 may be defined by the circumference of the pressurized area 330 and the length of the pressurized area 330. For example, the shape of the inflatable inner portion 238 may be determined by a rule (or formula) where the circumference of the pressurized area 330 is about or equal to twice the length of the pressurized area 330.

Accordingly, because the shape or form of the inflatable portion of the bladder may be variable depending upon the size of the finger, the need to have finger cuffs in different sizes may be eliminated. Therefore, a single-sized or one-size-fits-all type of finger cuff may be provided to accommodate large, medium, and small fingers, and to obtain accurate measurements. This further reduces product manufacturing costs.

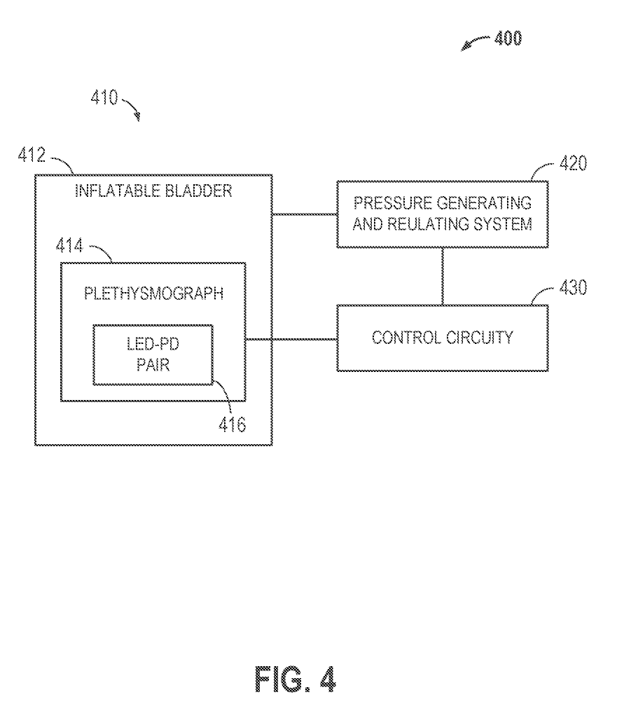

FIG. 4 is a block diagram illustrating an example environment 400 in which embodiments of the invention may be practiced. As shown, finger cuff assembly 410 may include an inflatable bladder 412 and a plethysmograph 414, with the inflatable bladder 412 surrounding and/or abutting the plethysmograph 414. The inflatable bladder 412 may be pneumatically connected to a pressure generating and regulating system 420. The pressure generating and regulating system 420 may generate, measure, and regulate pneumatic pressure that inflates or deflates an inner portion (e.g., inflatable inner portion 238, as previously described) of the bladder 412, and may include elements such as a pump, a valve, a sensor, control circuitry, and/or other suitable elements. When the inner portion of the bladder 412 is inflated, a pressure is applied to the patient's finger at a pressurized area (e.g., pressurized area 330 as previously described). The pressure applied to the finger may be the same as the pneumatic pressure in the inner portion of the bladder 412.

In one embodiment, the plethysmograph 414 may make continuous volumetric measurements (or plethysmogram) of arterial blood flows within the finger. In one embodiment, the plethysmograph 414 may include a LED-PD pair 416. The LED may be used to illuminate the finger skin and light absorption or reflection may be detected with the photodiode. Therefore, the plethysmogram may be generated based on the signal received from the photodiode.

The pressure generating and regulating system 420 and the plethysmograph 414 may be connected to a control circuitry 430. The control circuitry 430 may instruct the pressure generating and regulating system 420 to inflate or deflate the bladder 412 based on a pressure setting, may receive data from the plethysmograph 414, and may carry out necessary data manipulations.

It should be appreciated that aspects of the invention previously described may be implemented in conjunction with the execution of instructions by processors, circuitry, controllers, control circuitry, etc. As an example, control circuitry may operate under the control of a program, algorithm, routine, or the execution of instructions to execute methods or processes in accordance with embodiments of the invention previously described. For example, such a program may be implemented in firmware or software (e.g. stored in memory and/or other locations) and may be implemented by processors, control circuitry, and/or other circuitry, these terms being utilized interchangeably. Further, it should be appreciated that the terms processor, microprocessor, circuitry, control circuitry, circuit board, controller, microcontroller, etc., refer to any type of logic or circuitry capable of executing logic, commands, instructions, software, firmware, functionality, etc., which may be utilized to execute embodiments of the invention.

The various illustrative logical blocks, processors, modules, and circuitry described in connection with the embodiments disclosed herein may be implemented or performed with a general purpose processor, a specialized processor, circuitry, a microcontroller, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor may be a microprocessor or any conventional processor, controller, microcontroller, circuitry, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module/firmware executed by a processor, or any combination thereof. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A finger cuff assembly attachable to a patient's finger to be used in measuring the patient's blood pressure by a blood pressure measurement system, the finger cuff assembly comprising:
a plethysmograph including a light emitting diode (LED)—photodiode (PD) pair that aids in measuring the patient's blood pressure by the blood pressure measurement system;
an outer ring; and
a bladder contained within the outer ring, the bladder including an inflatable inner portion and a finger cavity, wherein the patient's finger with the plethysmograph are received and surrounded within the finger cavity of the bladder.

2. The finger cuff assembly of claim 1, wherein the inflatable inner portion of the bladder applies pneumatic pressure to a pressurized area of the patient's finger.

3. The finger cuff assembly of claim 2, wherein a length of the pressurized area is variable based on the size of the patient's finger.

4. The finger cuff assembly of claim 3, wherein a form of the inflatable inner portion is determined based on a circumference of the pressurized area and the length of the pressurized area.

5. The finger cuff assembly of claim 1, wherein the outer ring includes a rigid material.

6. The finger cuff assembly of claim 5, wherein the inflatable inner portion includes a flexible and non-elastic material.

7. The finger cuff assembly of claim 1, wherein the plethysmograph includes opaque and elastic material.

8. The finger cuff assembly of claim 1, wherein an interior of the plethysmograph includes an adhesive layer that is removably attached to the patient's finger to facilitate placement of the plethysmograph on or around the patient's finger.

9. A method to measure a patient's blood pressure by a blood pressure measurement system utilizing a finger cuff assembly, the finger cuff assembly comprising an outer ring, a plethysmograph having a light emitting diode (LED)—photodiode (PD) pair, a bladder contained within the outer ring, the bladder including an inflatable inner portion and a finger cavity, the method comprising:
placing the plethysmograph on a patient's finger such that the LED-PD pair aids in measuring the patient's blood pressure by the blood pressure measurement system; and
inserting the patient's finger with the plethysmograph through the finger cavity of the bladder such that the inflatable inner portion of the bladder surrounds and abuts against the plethysmograph on the patient's finger and the patient's finger.

10. The method of claim 9, further comprising applying, by the inflatable inner portion of the bladder, pneumatic pressure to a pressurized area of the patient's finger.

11. The method of claim 10, wherein a length of the pressurized area is variable based on the size of the patient's finger.

12. The method of claim 11, wherein a form of the inflatable inner portion is determined based on a circumference of the pressurized area and the length of the pressurized area.

13. The method of claim 9, wherein the outer ring includes a rigid material.

14. The method of claim 13, wherein the inflatable inner portion includes a flexible and non-elastic material.

15. The method of claim 9, wherein the plethysmograph includes opaque and elastic material.

16. The method of claim 9, wherein an interior of the plethysmograph includes an adhesive layer that is removably attached to the patient's finger to facilitate placement of the plethysmograph on the patient's finger.
